(19) United States
(12) Patent Application Publication
(10) Pub. No.: US 2011/0213365 A1
(43) Pub. Date: Sep. 1, 2011

(54) METHOD AND DEVICE FOR DEVITALIZING BIOLOGICAL TISSUE

(76) Inventors: Florian Eisele, Freiburg (DE); Matthias Voigtländer, Gomaringen (DE)

(21) Appl. No.: 13/122,787

(22) PCT Filed: Aug. 28, 2009
(86) PCT No.: PCT/EP2009/006255

§ 371 Date: May 10, 2011

(30) Foreign Application Priority Data
Oct. 7, 2008 (DE) 102008050635.4

(51) Int. Cl. A61B 18/18

(52) U.S. Cl. 606/41

(57) ABSTRACT

A device for devitalizing biological tissue and a method for controlling a device for devitalizing biological tissue. According to one embodiment, at the start of the devitalization, a low, first temperature is used and, at the end of the devitalization, an increased, second temperature is used. A device and method for operating a cooled HF (high frequency) ablation probe, wherein the temperature of the probe is primarily regulated via the cooling power supplied to the probe, is also described. According to another embodiment, the HF energy supply to the probe during treatment of a tissue region is operated first at a constant current and then at a constant voltage. According to another embodiment, the regulation of the cooling power is carried out depending on the impedance of the tissue region.

### FIELD OF THE INVENTION

The embodiments of the invention relate to a device for devitalizing biological tissue and a method for controlling a corresponding device.

### BACKGROUND

Electrosurgical devices, and particularly probes for devitalizing tissue (ablation probes) are known and comprise a probe body with at least one electrode for applying an HF (high frequency) voltage, and a cooling device. The HF voltage is generated via an HF generator.

In high frequency surgery, an alternating current is passed through the human body at a high frequency in order to selectively damage tissue. One application of high frequency surgery is the devitalizing of tumor tissue. High frequency surgery makes use of the thermal effects of heating, by which the devitalizing is achieved.

A distinction is drawn between a bipolar and a monopolar application of the HF current. In a monopolar application, the instrument of the electrosurgical device comprises only one electrode, while a second, neutral electrode is placed directly on the patient. The current flows from the electrode of the instrument to the neutral electrode in an inversely proportional relationship to the resistance of the tissue. The current density in the immediate vicinity of the electrode of the instrument is high enough for the described thermal effect to occur. With increasing distance from this electrode, the current density falls off in an inverse square relationship thereto. The devitalizing effect of the HF current is therefore spatially limited.

In a bipolar application, the instrument comprises two electrodes. For example, a probe tip can be configured as a first electrode, while a proximal section of the probe serves as the second electrode. The HF voltage is applied between the two electrodes, which are insulated from one another. The circuit is completed through the tissue situated between them. A current distribution field, concentrated around the probe, is produced.

It is self-evident that a high field density forms in the immediate vicinity of the instrument regardless of the type of application of the HF current (monopolar or bipolar). This field density can lead to dehydration and even carbonizing of the surrounding tissue. This effect is undesirable, at least in the devitalizing of tumors, since dehydrated or carbonized tissue has a strong insulating effect and hinders the treatment in deeper tissue regions. In addition, the body cannot readily decompose such carbonized tissue.

For this reason, a cooling device is used to cool the immediately adjacent tissue and prevent dehydration and/or carbonization of the adjacent tissue.

Depending on the size of the tumor, the devitalizing effect achieved with one instrument cannot be sufficient, in terms of volume and/or speed, to fully devitalize the tumor, including an oncological safety margin. In such a case, cluster electrodes and/or a plurality of ablation probes, which act like cluster electrodes, are used. Cluster electrodes can comprise two to four individual electrodes, which are arranged geometrically in relation to one another and which are supplied with an HF voltage. In application, both monopolar and bipolar versions are used, which are supplied by a generator and operated in parallel or in a multiplexed operation.

When a plurality of cooled application probes are operated in parallel at constant power by one generator, asymmetrical current distributions between the electrodes can arise at the start of the application due to varying starting conditions (impedance, heat capacity and heat conduction properties).

It is known to regulate electrosurgical instruments in a load-dependent manner such that the aforementioned dehydration or carbonization does not occur (see DE 42 33 467 A1), although such approaches cannot be used just as they are with cluster electrodes.

It can occur, for example, that a first electrode pair has a low current input (and only a little HF energy is imparted into the tissue), while a second electrode pair has a substantially higher current input. Given the constant cooling of the first electrode pair, due to the lowered temperature, the contact resistance increases such that the current input at this first electrode pair is reduced. In an unfavorable case, this can cause the current input at the second electrode pair to rise further. A mismatch then arises at the second electrode pair between the cooling power applied and the HF power. The tissue dries out such that the contact resistance at the second electrode also rises.

Using measurements of the contact resistance at the individual electrodes, one can determine whether the devitalization process or the coagulation process is considered to be concluded. With the described rise in the contact resistance at the first electrode pair (due to excessive cooling) and the second electrode pair (due to excessively high HF power), faulty estimations can arise such that the coagulation process can be falsely assessed as being concluded. It is obvious that the lack, or excessively weak application, of an HF current to the first electrode negatively influences the overall result of the intervention. The brief application of the HF current at a high voltage at the second electrode pair can also cause tissue remote from the electrodes to not be devitalized.

If the devitalization process is not interrupted, due to the rise in contact resistance at the first electrode pair and the second electrode pair, then a self-regulating effect arises. Since the contact resistance rises substantially more strongly at the second electrode pair than at the first electrode pair, the HF power distribution becomes displaced in favor of the first electrode pair. Tissue fluid can diffuse back again to the extent that “only” dehydration takes place at the second electrode pair. The contact resistance at the second electrode pair falls, whereas that at the first electrode pair rises, possibly due to dehydration of the tissue. The system therefore tends to oscillate back and forth between the two states, with the HF power at the individual electrode pairs rising and falling back again. A precondition for this is that no irreversible effects occur at the contact tissue.

However, the system tends to overshoot, particularly when there are relatively large distances between the electrode clusters, since too much cooling energy at one probe is not compensated for by the diffusion of warmth from the other probe.

Given the multiple operation of the probes in clusters or arrays, an excessive rise in impedance is prevented because the ablation probes, probes or probe pairs are operated one after the other using a particular algorithm. However, this has the disadvantage that efficient devitalization by continuous energy input into the tissue is not possible. Even if only one ablation probe is used, conventional control algorithms are not sufficient to prevent excessive cooling or overheating of the directly adjacent tissue. It is known, for example, to perform regulation based on the gradient of the current fall (dI/dt) or the impedance rise (dZ/dt). This type of regulation, however, is insufficient.

### SUMMARY

It is therefore an object of the embodiments of the invention to improve the control of cooled ablation probes both in single operation and in cluster operation. In particular, a device for devitalizing biological tissue is provided, which enables devitalization of predefined tissue sections that is both reliable and reduces the burden on the patient. It is a further object to define a corresponding method.

This aim is achieved with a device for devitalizing biological tissue, comprising at least one ablation probe, which has cooling devices that are configured and arranged such that tissue regions close to the ablation probe can be cooled by the cooling power thereof, and which has electrode units arranged and configured in such a way that an HF treatment current generated by an HF generator can be conducted into the tissue, and a regulation unit, configured and connected to the cooling devices and to the HF generator such that, at the start of the devitalization, a low, first temperature is used and, at the end of the devitalization, an increased, second temperature is used.

The essence of the embodiments of the invention is based on the recognition that tissue change due to excessive cooling is essentially reversible, whereas the dehydration and/or carbonization of tissue due to excessive heating cannot, or can only very slowly, be regenerated. It is therefore useful to intervene by using the cooling device in a regulating and timely manner i.e., from the start. According to the embodiments of the invention, therefore, at the start of the devitalization, cooling is much more strongly applied, whereas toward the end of the process, the temperature is increased. The temperature can be controlled either by regulating the cooling power or by regulating the ratio between HF power and cooling power. At the first temperature, sufficient expansion of the coagulation zone is ensured by enabling a current input into the further removed tissue regions. After reaching the desired coagulation radius, the tissue adjacent to the ablation probe is devitalized on application of the second temperature.

It is advantageous to operate the device such that the first temperature is selected to be close to the freezing point of the tissue being treated, particularly between 1° C. and 8° C. However, the regulation unit should be configured such that icing of the tissue is prevented. On icing of the tissue, the specific resistance increases, which is the reason why a low current input into the tissue can arise.

The regulation unit can be configured for setting the first temperature by defining a first treatment current and for setting the second temperature by defining a first treatment voltage and, in each case, a corresponding adjustment of the cooling power. Since the resistance change in a large region close to the first temperature is negative, a stable regulation of the coagulation process can be ensured on operation with an essentially constant first treatment current. Since the resistance change in a large region close to the second temperature is positive, in this second operating mode, a stable setting of the coagulation process with an essentially constant treatment voltage can be achieved.

The regulation unit can comprise a constant current source for setting the first treatment current.

The regulation unit can comprise a constant voltage source for setting the first treatment voltage.

The regulation unit can be set such that a smooth transition takes place from current regulation to voltage regulation.

In order to determine the first and/or second temperature, the regulation unit can comprise an impedance measurement apparatus for measuring the impedance between the electrode unit and the surrounding tissue. For example, the impedance between the neutral electrode and the application electrode can be measured. Alternatively, the impedance between the electrode pairs, which are supplied with a voltage to apply the HF current, can be measured. The individual electrodes can also be constructed in multiple parts such that a measurement is made between the electrode parts. The impedance measurement can advantageously be processed to draw conclusions about the conditions, particularly the prevailing temperature, existing at the ablation probe.

The device can comprise a plurality of ablation probes and regulation units, wherein a higher-level regulation/control unit is provided and configured such that the respective ablation probes are controllable together by associated regulation units. Particularly, it is advantageous to prevent vapor formation and dehydration at the start of the devitalization or coagulation process, when operating a plurality of ablation probes, by operating the individual ablation probes at a first lower temperature.

The problem set out above is also solved by a method for regulating a device for devitalizing biological tissue, wherein the device comprises at least one ablation probe, which comprises cooling devices that are configured and arranged such that, due to the cooling power thereof, tissue regions close to the ablation probe can be cooled, and said device comprises electrode units configured and arranged such that a treatment current can be conducted into the tissue, wherein at the start of the devitalization, a low, first temperature is used and, at the end of the devitalization, an increased, second temperature is used.

Further advantageous embodiments are contained in the dependent claims. The advantages achieved with the disclosed method essentially correspond to those of the previously described device.

### BRIEF DESCRIPTION OF THE DRAWINGS

Embodiments of the invention will now be described in more detail with reference to the drawings, wherein:

FIG. 1 illustrates the essential components of the device for devitalizing biological tissue according to an embodiment of the invention;

FIG. 2 illustrates a plurality of ablation probes with the regulation/control unit according to an embodiment of the invention; and

FIG. 3 illustrates a graph illustrating the dependence of the specific resistance of biological tissue on temperature.

### DETAILED DESCRIPTION

In the following description, the same reference signs are used for identical parts and parts acting in an identical manner.

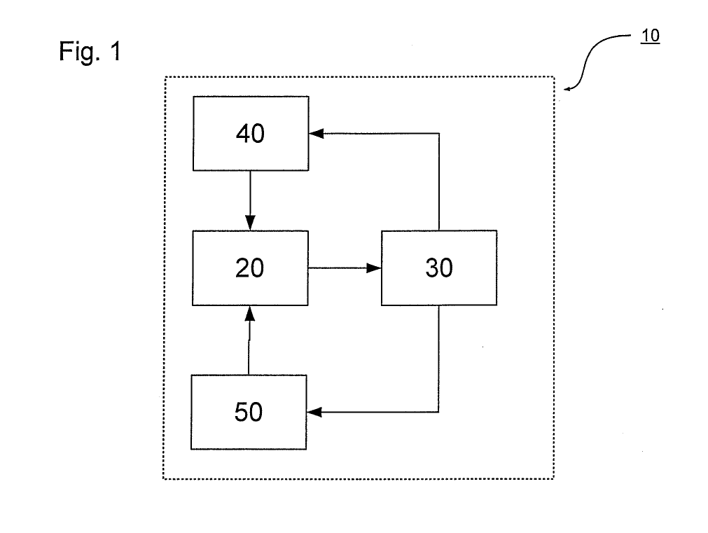

A first exemplary embodiment of the device 10 for devitalizing biological tissue comprises an ablation probe 20 and a regulation/control unit 30 (see FIG. 1). In order to supply an HF voltage to the ablation probe 20, said probe is connected to an HF generator 50. There is also a fluid connection to a coolant source 40, which supplies coolant for cooling the ablation probe 20. The regulation/control unit 30 controls or regulates the coolant source 40 such that the ablation probe is cooled with a pre-defined cooling power P_{K}. The regulation/control unit 30 also controls or regulates the HF generator 50 such that the ablation probe 20 outputs a pre-defined HF power P_{HF} to the tissue.

In order to suitably regulate the HF power P_{HF} and the cooling power P_{K}, the regulation/control unit 30 receives measurement signals from the ablation probe 20.

In the exemplary illustrated embodiment, a specific conductivity or a specific resistance ρ of the tissue is to be measured.

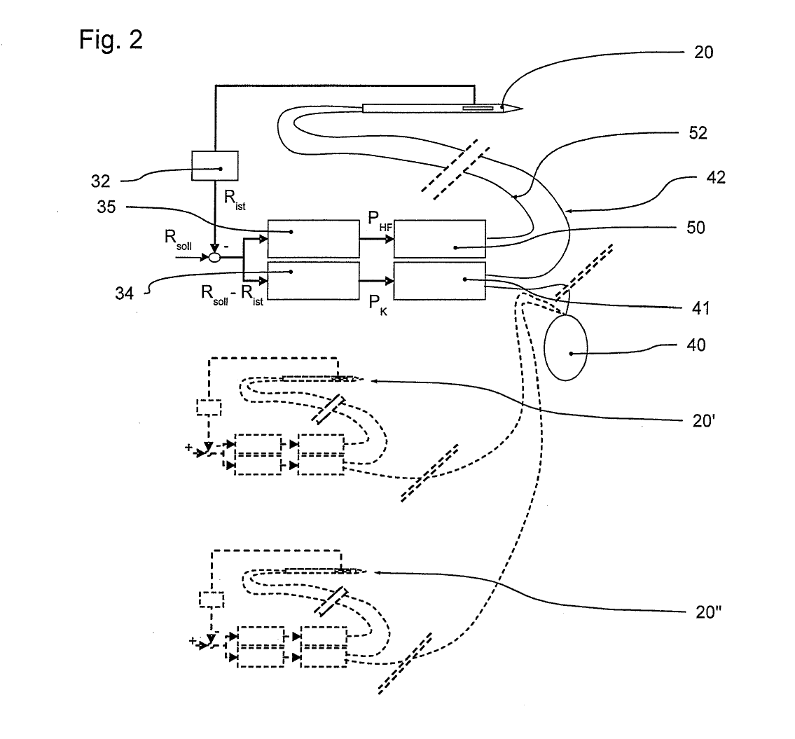

As indicated schematically in FIG. 2, the device 10 comprises a measurement device 32 for this purpose, which detects an actually existing impedance Rᵢₛₜ. This actual impedance Rᵢₛₜ can be measured between one or more electrode pairs. It is also conceivable for one electrode to be constructed in two or more parts and the resistance to be measured is taken between the individual parts of the electrode.

The specific conductivity of the tissue reveals information about the temperature of the tissue and about its condition thereof (e.g., frozen, dehydrated or carbonized).

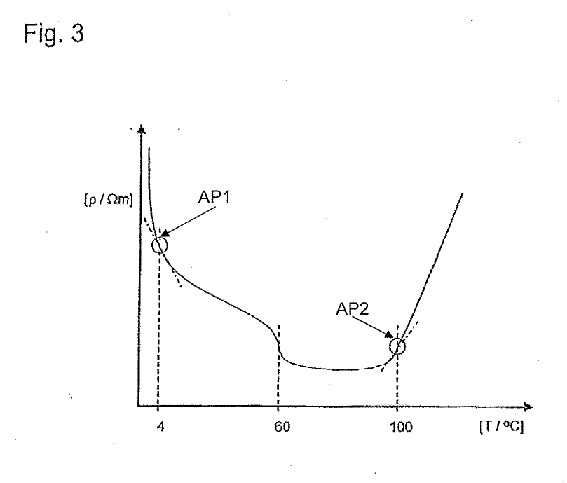

The specific conductivity of the tissue is influenced by the energy introduced or extracted during the course of the treatment, that is, by the HF power P_{HF} and the cooling power P_{K}. The specific conductivity therefore falls (i.e., specific conductivity=1/ρ) if the tissue temperature approaches the freezing point (see FIG. 3). Conversely, if high temperatures occur, particularly greater than or equal to 100° C., vapor formation and dehydration of the tissue takes place. Thus, the specific conductivity falls significantly if the temperature of the tissue is greater than or equal to 100° C.

The regulation/control unit 30 according to the embodiment of the invention uses the HF power P_{HF} and cooling power P_{K} adjustment variables to coagulate the tissue at a predetermined temperature. In particular, it is part of the embodiment of the invention to achieve a predetermined temperature gradient in the tissue being treated.

In abstract terms, a target impedance Rₛₒₗₗ is compared with the measured actual impedance Rᵢₛₜ and the adjustment variables are set according to the desired result. In order to regulate the HF power P_{HF} and cooling power P_{K}, the regulation/control unit 30 comprises a cooling power regulator 34 and an HF power regulator 35.

The HF power regulator 35 can, for example, control the HF generator 50, which supplies the ablation probe 20 with the HF current via a line 52. The cooling power regulator 34 can regulate the coolant supply from the coolant source 40 by regulating a valve 41, and can thus set the cooling power P_{K}.

According to the invention, the device 10 for devitalizing tissue has at least two operating states. In a first operating state, the ablation probe 20 is operated at a first operating point AP1 and, in a second operating state, the ablation probe 20 is operated at a second operating point AP2. The first operating point AP1 is characterized by a first low temperature, particularly in the interval between 2° C. and 10° C. and the second operating point AP2 is characterized by a significantly higher, second temperature, particularly in the range from 80° C. to 110° C. The temperature data relate particularly to the tissue immediately adjacent to the ablation probe 20 and/or to the temperature at the outer sleeves of the ablation probe 20.

At the first operating point AP1, a current input into the further removed tissue regions is enabled due to the cooling of the tissue round the probe surface. The thermal effect for the devitalizing of tissue therefore occurs in regions that are relatively far removed from the applying ablation probe 20. In the event of a deviation around the first operating point AP1, irreversible changes do not take place in the tissue due to dehydration (cf., denaturing at the second operating point AP2). Freezing of the adjacent tissue would be a reversible change, which is relatively not problematic. In particular, the tissue can be thawed out again within a few seconds. There is, therefore, a temperature distribution wherein a relatively low temperature exists in the direct vicinity (e.g., the first temperature) and the temperature increases with increasing distance, up to a maximum value. After reaching the maximum value, the temperature falls again to body temperature.

After reaching the desired coagulation radius, the device 10 assumes the second operating state and regulates the adjustment variables such that the second operating point AP2 is reached. It is also conceivable to undertake a continuous transition from the regulation at the first operating point to the regulation at the second operating point AP2. Thus, the coagulation radius can be reduced step-by-step. Since the specific resistance (Δρ/ΔT) is negative in a large interval around the first operating point AP1, during operation at constant current, a stable operating point can be realized.

At the same time, the change of specific resistance (Δρ/ΔT) is positive in a large interval around the second operating point AP2, for which reason, a stable second operating point can be realized during operation at constant voltage.

Referring to FIG. 3, which shows the specific resistance of biological tissue as a function of the prevailing temperature (the abscissa represents the temperature in ° C., while the ordinate shows the specific resistance in Ohm-meters), it is apparent that the illustrated operating points AP1 and AP2 show a corresponding resistance change. It is also apparent that a first operating point AP1 at approximately 4° C. is particularly advantageous, since at a lower temperature, the specific resistance rises sharply. This operating point AP1 can therefore easily be detected.

It is also apparent that the second operating point AP2 is also very characteristic at a temperature of approximately 100° C., since at higher temperatures, the specific resistance also rises sharply. The finding of the characteristic operating points AP1 and AP2 and the maintenance thereof is therefore relatively easily achieved.

In a further exemplary embodiment, as shown in FIG. 2, a plurality of ablation probes 20, 20′, 20″ are simultaneously operated with a plurality of regulation/control units 30, in order to deactivate a large tumor region. Insofar as the individual ablation probes 20, 20′, 20″ are similarly regulated and controlled as described above, advantageous devitalization of the tissue takes place. In particular, complete devitalization of large tissue sections can be achieved in a minimally damaging manner for the patient.

1-15. (canceled)

16. A device for devitalizing biological tissue, said device comprising:
at least one ablation probe having cooling devices configured and arranged such that tissue regions close to the at least one ablation probe can be cooled by the cooling power thereof, and which has electrode units arranged and configured such that a high frequency (HF) treatment current generated by an HF generator can be conducted into the tissue, and
a regulation unit configured and connected to the cooling devices and to the HF generator such that, at the start of the devitalization, a low, first temperature is used to prevent an irreversible change to the tissue directly adjacent to the at least one ablation probe and, at the end of the devitalization, an increased, second temperature is used,
wherein the regulation unit is configured for setting the first temperature by defining a first treatment current and for setting the second temperature by defining a first treatment voltage and, in each case, a corresponding adjustment of the cooling power.

17. The device of claim 16, wherein the regulation unit is configured so that the first temperature is set to prevent icing of the tissue.

18. The device of claim 16, wherein the regulation unit comprises a constant current source for setting the first treatment current.

19. The device of claim 16, wherein the regulation unit comprises a constant voltage source for setting the first treatment voltage.

20. The device of claim 16, wherein the regulation unit is configured such that a smooth transition is carried out from current regulation to voltage regulation.

21. The device of claim 16, wherein to determine the first and second temperature, the regulation unit comprises an impedance measurement apparatus for measuring an impedance between the electrode unit and the surrounding tissue.

22. The device of claim 16, wherein to determine the first or second temperature, the regulation unit comprises an impedance measurement apparatus for measuring an impedance between the electrode unit and the surrounding tissue.

23. The device of claim 16, further comprising a plurality of ablation probes and regulation units, wherein a higher-level regulation/control unit is configured such that the respective ablation probes are controllable together by associated regulation units.

24. A method of controlling a device for devitalizing biological tissue, wherein the device comprises at least one ablation probe that comprises cooling devices that are configured and arranged such that, due to the cooling power thereof, tissue regions close to the ablation probe can be cooled, and has electrode units configured and arranged such that an HF treatment current can be conducted into the tissue, said method comprising:
at the start of the devitalization, applying a low, first temperature; and
at the end of the devitalization, applying an increased, second temperature,
wherein the first temperature is set by pre-setting a first treatment current and the second temperature is set by pre-setting a first treatment voltage and, in each case, by appropriately setting the cooling power.

25. The method of claim 24, wherein the first temperature is set to prevent icing of the tissue.

26. The method of claim 24, wherein the first treatment current is generated as a constant current and the first treatment voltage is generated as a constant voltage.

27. The method of claim 24, wherein the first treatment current is generated as a constant current or the first treatment voltage is generated as a constant voltage.

28. The method of claim 24, wherein a smooth transition is carried out from current regulation to voltage regulation.

29. The method of claim 24, wherein an impedance is measured between the electrode units and the tissue to determine the first and second temperature.

30. The method of claim 24, wherein an impedance is measured between the electrode units and the tissue to determine the first or second temperature.

31. The method of claim 24, wherein a plurality of ablation probes are regulated individually for setting the first and second temperature and together for carrying out a treatment procedure.
